SEQUENCE LISTING (5 sequences)

SEQ ID NO: 1 - protein, 299 aa, Homo sapiens
MLESHLIIYFLLAVIQFLLGIFTNGIIVVVNGIDLIKHRKMAPLDLLLSCLAVSRIFLQLFIFYVNVIVIFFIEFIMCSANCAILLFINELELWLATWLGVFYCAKVASVRHPLFIWLKMRISKLVPWMILGSLLYVSMICVFHSKYAGFMVPYFLRKFFSQNATIQKEDTLAIQIFSFVAEFSVPLLIFLFAVLLLIFSLGRHTRQMRNTVAGSRVPGRGAPISALLSILSFLILYFSHCMIKVFLSSLKFHIRRFIFLFFILVIGIYPSGHSLILILGNPKLKQNAKKFLLHSKCCQ

SEQ ID NO: 2 - protein, 307 aa, Homo sapiens
MLRVVEGIFIFVVVSESVFGVLGNGFIGLVNCIDCAKNKLSTIGFILTGLAISRIFLIWIIITDGFIQIFSPNIYASGNLIEYISYFWVIGNQSSMWFATSLSIFYFLKIANFSNYIFLWLKSRTNMVLPFMIVFLLISSLLNFAYIAKILNDYKMKNDTVWDLNMYKSEYFIKQILLNLGVIFFFTLSLITCIFLIISLWRHNRQMQSNVTGLRDSNTEAHVKAMKVLISFIILFILYFIGMAIEISCFTVRENKLLLMFGMTTTAIYPWGHSFILILGNSKLKQASLRVLQQLKCCEKRKNLRVT

SEQ ID NO: 3 - protein, 317 aa, Homo sapiens
MGGVIKSIFTFVLIVEFIIGNLGNSFIALVNCIDWVKGRKISSVDRILTALAISRISLVWLIFGSWCVSVFFPALFATEKMFRMLTNIWTVINHFSVWLATGLGTFYFLKIANFSNSIFLYLKWRVKKVVLVLLLVTSVFLFLNIALINIHINASINGYRRNKTCSSDSSNFTRFSSLIVLTSTVFIFIPFTLSLAMFLLLIFSMWKHRKKMQHTVKISGDASTKAHRGVKSVITFFLLYAIFSLSFFISVWTSERLEENLIILSQVMGMAYPSCHSCVLILGNKKLRQASLSVLLWLRYMFKDGEPSGHKEFRESS

SEQ ID NO: 4 - protein, 309 aa, Homo sapiens
MITFLPIIFSSLVVVTFVIGNFANGFIALVNSIEWFKRQKISFADQILTALAVSRVGLLWVLLLNWYSTVLNPAFNSVEVRTTAYNIWAVINHFSNWLATTLSIFYLLKIANFSNFIFLHLKRRVKSVILVMLLGPLLFLACHLFVINMNEIVRTKEFEGNMTWKIKLKSAMYFSNMTVTMVANLVPFTLTLLSFMLLICSLCKHLKKMQLHGKGSQDPSTKVHIKALQTVISFLLLCAIYFLSIMISVWSFGSLENKPVFMFCKAIRFSYPSIHPFILIWGNKKLKQTFLSVFWQMRYWVKGEKTSSP

SEQ ID NO: 5 - protein, 299 aa, Homo sapiens
MITFLYIFFSILIMVLFVLGNFANGFIALVNFIDWVKRKKISSADQILTALAVSRIGLLWALLLNWYLTVLNPAFYSVELRITSYNAWVVTNHFSMWLAANLSIFYLLKIANFSNLLFLHLKRRVRSVILVILLGTLIFLVCHLLVANMDESMWAEEYEGNMTGKMKLRNTVHLSYLTVTTLWSFIPFTLSLISFLMLICSLCKHLKKMQLHGEGSQDLSTKVHIKALQTLISFLLLCAIFFLFLIVSVWSPRRLRNDPVVMVSKAVGNIYLAFDSFILIWRTKKLKHTFLLILCQIRC

(19) United States
(12) Patent Application Publication
(10) Pub. No.: US 2025/0052739 A1
(43) Pub. Date: Feb. 13, 2025

(54) REDUCTION OF BITTER TASTE OF PLANT PROTEINS AND RELATED ASSAYS AND SCREENING METHODS

(71) Applicant: Firmenich Incorporated, Plainsboro, NJ (US)

(72) Inventors: Guy Servant, Plainsboro, NJ (US); Mark E. Williams, Plainsboro, NJ (US); Joseph R. Fotsing, Plainsboro, NJ (US); Lan Zhang, Plainsboro, NJ (US)

(21) Appl. No.: 18/717,767

(22) PCT Filed: Dec. 5, 2022
(86) PCT No.: PCT/US2022/080945

§ 371 Date: Jun. 7, 2024

Related U.S. Application Data
(60) Provisional application No. 63/286,882, filed on Dec. 7, 2021.

(30) Foreign Application Priority Data
Feb. 1, 2022 (EP) 22154411.7

(51) Int. Cl. G01N 33/50; G01N 33/566

(52) U.S. Cl. CPC G01N 33/5008; G01N 33/566; G01N 2333/415; G01N 2500/10

(57) ABSTRACT

The present disclosure generally provides methods for identifying compounds that attenuate the bitter taste of plant proteins. In some particular aspects, the disclosure provides methods of identifying compounds that attenuate the bitter taste of plant proteins by determining whether the compounds modulate certain T2R bitter taste receptors. In some other aspects, the disclosure provides uses of such identified compounds to attenuate the bitter taste of an ingestible composition that comprises one or more plant proteins. In some further aspects, the disclosure provides flavored products, such as food or beverage products, that incorporate such ingestible compositions. Such food products include meat and dairy analogues.

### TECHNICAL FIELD

The present disclosure generally provides methods for identifying compounds that attenuate the bitter taste of plant proteins. In some particular aspects, the disclosure provides methods of identifying compounds that attenuate the bitter taste of plant proteins by determining whether the compounds modulate certain T2R bitter taste receptors. In some other aspects, the disclosure provides uses of such identified compounds to attenuate the bitter taste of an ingestible composition that comprises one or more plant proteins. In some further aspects, the disclosure provides flavored products, such as food or beverage products, that incorporate such ingestible compositions. Such food products include meat and dairy analogues.

### DESCRIPTION OF RELATED ART

The use of plant proteins in food and beverage products has gained increasing acceptance over the last few years, and is expected to grow considerably in the coming decades. The reasons for this growth include potential health benefits of eating a diet richer in plant-based materials, as well as an increased focus on sustainable consumption. But consumers are unlikely to substitute meat-based products with plant-based products if the plant-based products fail to provide taste and texture profiles that are perceived as desirable. Nearly all food and beverage products that use appreciable amounts of plant-based protein have a detectable bitter taste. This limits the broad acceptance of such products in the marketplace, and highlights the need to develop strategies to mask or block the perception of such bitterness.

In humans, bitter taste is mediated by a family of 25 G protein-coupled receptors (GPCRs) that are expressed at the surface of taste receptor cells. Most bitter-tasting foods or beverages bind to a few of these 25 receptors. Therefore, blocking bitterness at the receptor level can be quite complicated. For this reason, bitter masking agents are often used instead. These materials do not block the agonism of the T2R taste receptors, but instead coat the tongue and mouth and prevent physical contact of the bitter-tasting compounds with the taste receptor cells. Of course, such masking agents also prevent physical contact with sweet and umami taste receptors, which, when activated, tend to enhance the perceived desirability of food or beverage products. So, while such bitter maskers may reduce bitter taste, their use often makes the product less tasty overall, or requires the addition of sugars, glutamates, and salt to overcome this taste-dulling effect. Thus, there is a continuing need to develop strategies for identifying compounds that can selectively block the bitter taste of plant-based proteins at the receptor level and for using such compounds in conjunction with food and beverage products that include plant-based proteins.

### SUMMARY

The present disclosure relates to the discovery that certain of the 25 T2R taste receptors are principally responsible for the human perception of the bitter taste of plant-based proteins, such as pea protein. Thus, the methods disclosed herein provide a means of screening for and identifying compounds that can selectively block one or more of these certain T2R receptors. The disclosure also describes the use of such compounds in combination with food and beverage products that contain plant-based proteins.

In a first aspect, the disclosure provides methods of identifying compounds that reduce a bitter taste of a plant protein, the methods comprising: (a) introducing a test compound to a taste receptor, which comprises a polypeptide sequence of SEQ ID NO: 1, SEQ ID NO: 2, SEQ ID NO: 3, SEQ ID NO: 4, SEQ ID NO: 5, a functional fragment of any of the foregoing, or a polypeptide sequence whose sequence is at least 90% equivalent to any of the foregoing; and (b) measuring a response of the taste receptor to the test compound by comparing an activity of the taste receptor in the presence and the absence of the test compound. In some embodiments, the methods further comprise: (c) identifying an active test compound that reduces the bitter taste of the plant protein based on the measured response; and, optionally, (d) selecting the active test compound as a compound that reduces the bitter taste of the plant protein.

In a second aspect, the disclosure provides methods of identifying compounds that reduce a bitter taste of a plant protein, the methods comprising: (a) introducing a test compound to a taste receptor in the presence of a plant protein, wherein the taste receptor comprises a polypeptide sequence of SEQ ID NO: 1, SEQ ID NO: 2, SEQ ID NO: 3, SEQ ID NO: 4, SEQ ID NO: 5, a functional fragment of any of the foregoing, or a polypeptide sequence whose sequence is at least 90% equivalent to any of the foregoing; and (b) measuring a response of the taste receptor to the test compound by comparing an activity of the taste receptor in the presence and the absence of the test compound. In some embodiments, the methods further comprise: (c) identifying an active test compound that reduces the bitter taste of the plant protein based on the measured response; and, optionally, (d) selecting the active test compound as a compound that reduces the bitter taste of the plant protein.

In a third aspect, the disclosure provides uses of an identified or selected active test compound of any embodiments of the first or second aspect to reduce a bitter taste of a plant protein in an ingestible composition. In some embodiments, the plant protein is pea protein.

In a fourth aspect, the disclosure provides methods of reducing a bitter taste of a plant protein in an ingestible composition, the method comprising introducing to an ingestible composition comprising a plant protein an identified or selected active test compound of any embodiments of the first or second aspect. In some embodiments, the plant protein is pea protein.

In a fifth aspect, the disclosure provides ingestible compositions comprising a plant protein and an identified or selected active test compound of any embodiments of the first or second aspect. In some embodiments, the ingestible composition is a naturally occurring composition. In some embodiments, the ingestible composition is a non-naturally occurring composition. In some embodiments, the plant protein is pea protein.

In a sixth aspect, the disclosure provides flavored products comprising an ingestible composition of the fifth aspect. In some embodiments, the flavored products are food products or beverage products, such as a meat analogue product or a dairy analogue product.

Further aspects, and embodiments thereof, are set forth below in the Detailed Description, the Drawings, the Abstract, and the Claims.

### FIGURES

FIG. 1: Cells expressing different bitter taste receptors were stimulated with controls or ethyl acetate fractions derived from Pea and Soy extracts. Pea and Soy fractions activate hT2R10, hT2R14 or hT2R61.

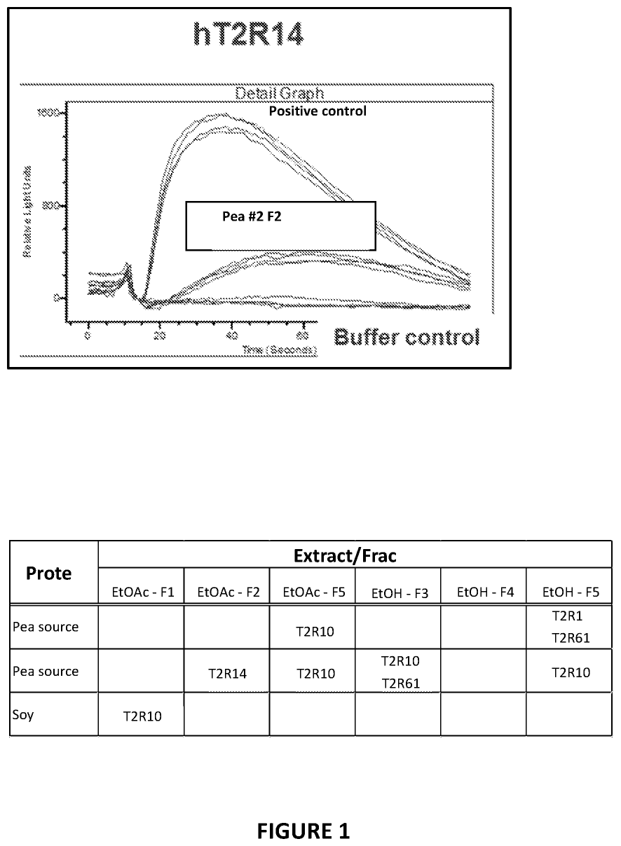

FIG. 2: Cpd A inhibits hT2R10, hT2R14 and hT2R61 in the assay. Cpd B inhibits hT2R10 and hT2R61 in the assay.

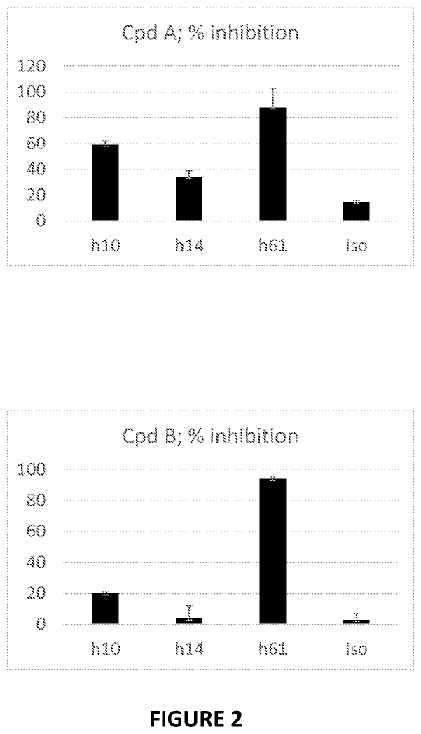

FIG. 3: Cpd A and Cpd B attenuate bitterness of Pea protein

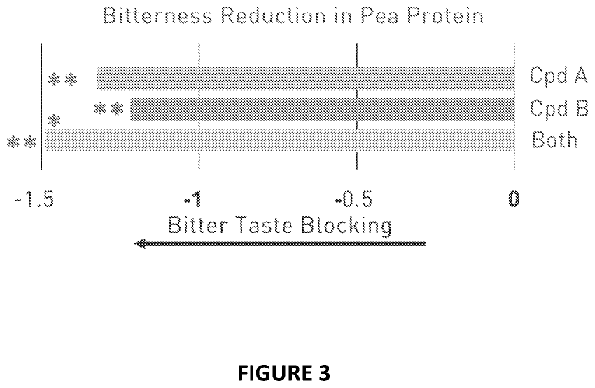

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

A “bitter compound” or a “bitter tastant” refers to a compound that elicits a detectable bitter taste sensation in a subject or that agonizes one or more T2R bitter taste receptors. A “bitter blocker” refers to a compound that inhibits the binding of a bitter compound to one or more T2R bitter taste receptors. In the context of this disclosure, the terms “inhibit” or “block” do not necessarily require complete inhibition or blocking, but encompass any reduction in the binding activity of the bitter compound.

A “functional fragment” refers to a portion of a polypeptide sequence to which the umami compound or the test compound binds. Polypeptide sequences often contain certain amino acids that do not actively participate in binding, but which may serve other purposes. In some instances, these non-functioning parts of the polypeptide sequence can be removed or partially replaced, while leaving the functional portion of the sequence intact. These modified proteins are said to comprise a functional fragment of the original polypeptide sequence.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A1 and A2, then one or more members of the class can be present concurrently.

Other terms are defined in other portions of this description, even though not included in this subsection.

### Polypeptide Sequences of Taste Receptors

The present disclosure provides certain polypeptide sequences that are useful in the simulating in vitro the response that certain human taste receptors would exhibit towards such compounds when ingested orally.

In some embodiments, the disclosure provides a polypeptide sequence of SEQ ID NO: 1 or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 1 (hT2R1), starting with its N-terminus, is: MLESHLIIYFLLAVIQFLLGIFTNGIIVVVNGIDLIKHRKMAPLDLLL SCLAVSRIFLQLFIFYVNVIVIFFIEFIMCSANCAILLFINELELWLATWLGVFYCAKVA SVRHPLFIWLKMRISKLVPWMILGSLLYVSMICVFHSKYAGFMVPYFLRKFFSQNATI QKEDTLAIQIFSFVAEFSVPLLIFLFAVLLLIFSLGRHTRQMRNTVAGSRVPGRGAPISA LLSILSFLILYFSHCMIKVFLSSLKFHIRRFIFLFFILVIGIYPSGHSLILILGNPKLKQNAK KFLLHSKCCQ, using the standard single-letter amino acid codes.

In some embodiments, the disclosure provides a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 2 (hT2R10), starting with its N-terminus, is: MLRVVEGIFIFVVVSESVFGVLGNGFIGLVNCIDCAKNKLSTIGFILT GLAISRIFLIWIIITDGFIQIFSPNIYASGNLIEYISYFWVIGNQSSMWFATSLSIFYFLKIA NFSNYIFLWLKSRTNMVLPFMIVELLISSLLNFAYIAKILNDYKMKNDTVWDLNMYK SEYFIKQILLNLGVIFFFTLSLITCIFLIISLWRHNRQMQSNVTGLRDSNTEAHVKAMK VLISFIILFILYFIGMAIEISCFTVRENKLLLMFGMTTTAIYPWGHSFILILGNSKLKQAS LRVLQQLKCCEKRKNLRVT, using the standard single-letter amino acid codes.

In some other embodiments, the disclosure provides a polypeptide sequence of SEQ ID NO: 3, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 3 (hT2R14), starting with its N-terminus, is: MGGVIKSIFTFVLIVEFIIGNLGNSFIALVNCIDWVKGRKISSVDRIL TALAISRISLVWLIFGSWCVSVFFPALFATEKMFRMLTNIWTVINHFSVWLATGLGTF YFLKIANFSNSIFLYLKWRVKKVVLVLLLVTSVFLFLNIALINIHINASINGYRRNKTCS SDSSNFTRFSSLIVLTSTVFIFIPFTLSLAMFLLLIFSMWKHRKKMQHTVKISGDASTKA HRGVKSVITFFLLY AIFSLSFFISVWTSERLEENLIILSQVMGMAYPSCHSCVLILGNKK LRQASLSVLLWLRYMFKDGEPSGHKEFRESS, using the standard single-letter amino acid codes.

In some other embodiments, the disclosure provides a polypeptide sequence of SEQ ID NO: 4, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 4 (hT2R61), starting with its N-terminus, is: MITFLPIIFSSLVVVTFVIGNFANGFIALVNSIEWFKRQKISFADQILT ALAVSRVGLLWVLLLNWYSTVLNPAFNSVEVRTTAYNIWAVINHFSNWLATTLSIFY LLKIANFSNFIFLHLKRRVKSVILVMLLGPLLFLACHLFVINMNEIVRTKEFEGNMTW KIKLKSAMYFSNMTVTMVANLVPFTLTLLSFMLLICSLCKHLKKMQLHGKGSQDPS TKVHIKALQTVISFLLLCAIYFLSIMISVWSFGSLENKPVFMFCKAIRFSYPSIHPFILIW GNKKLKQTFLSVFWQMRYWVKGEKTSSP, using the standard single-letter amino acid codes.

In some other embodiments, the disclosure provides a polypeptide sequence of SEQ ID NO: 5, or a functional fragment thereof, or a polypeptide sequence whose sequence is at least 90% equivalent, or at least 95% equivalent, or at least 97% equivalent, to either of the foregoing. For purposes of clarity and to avoid any confusion, the polypeptide sequence corresponding to SEQ ID NO: 5 (hT2R67), starting with its N-terminus, is: MITFLYIFFSILIMVLFVLGNFANGFIALVNFIDWVKRKKISSADQIL TALAVSRIGLLWALLLNWYLTVLNPAFYSVELRITSYNAWVVTNHFSMWLAANLSI FYLLKIANFSNLLFLHLKRRVRSVILVILLGTLIFLVCHLLVANMDESMWAEEYEGN MTGKMKLRNTVHLSYLTVTTLWSFIPFTLSLISFLMLICSLCKHLKKMQLHGEGSQD LSTKVHIKALQTLISFLLLCAIFFLFLIVSVWSPRRLRNDPVVMVSKAVGNIYLAFDSFI LIWRTKKLKHTFLLILCQIRC, using the standard single-letter amino acid codes.

The polypeptide sequences of the foregoing aspects and embodiments can be present in any suitable composition. In some embodiments, one or more polypeptide sequences of the foregoing aspects and embodiments is present in a non-naturally occurring composition, such as an in vitro assay. In some further such embodiments, the polypeptide sequences of the foregoing aspects and embodiments are expressed on the surface of cells, such as on the cells of a eukaryotic cell line.

### Screening Methods

In certain aspects, the disclosure provides methods of identifying compounds that reduce a bitter taste of a plant protein, the methods comprising: (a) introducing a test compound to a taste receptor, which comprises a polypeptide sequence of SEQ ID NO: 1, SEQ ID NO: 2, SEQ ID NO: 3, SEQ ID NO: 4, SEQ ID NO: 5, a functional fragment of any of the foregoing, or a polypeptide sequence whose sequence is at least 90% equivalent to any of the foregoing; and (b) measuring a response of the taste receptor to the test compound by comparing an activity of the taste receptor in the presence and the absence of the test compound. In some embodiments, the methods further comprise: (c) identifying an active test compound that reduces the bitter taste of the plant protein based on the measured response; and, optionally, (d) selecting the active test compound as a compound that reduces the bitter taste of the plant protein.

In certain aspects, the disclosure provides methods of identifying compounds that reduce a bitter taste of a plant protein, the methods comprising: (a) introducing a test compound to a taste receptor in the presence of a plant protein, wherein the taste receptor comprises a polypeptide sequence of SEQ ID NO: 1, SEQ ID NO: 2, SEQ ID NO: 3, SEQ ID NO: 4, SEQ ID NO: 5, a functional fragment of any of the foregoing, or a polypeptide sequence whose sequence is at least 90% equivalent to any of the foregoing; and (b) measuring a response of the taste receptor to the test compound by comparing an activity of the taste receptor in the presence and the absence of the test compound. In some embodiments, the methods further comprise: (c) identifying an active test compound that reduces the bitter taste of the plant protein based on the measured response; and, optionally, (d) selecting the active test compound as a compound that reduces the bitter taste of the plant protein.

The plant protein can be any suitable protein extracted from a plant source. The plant source is typically a plant whose proteins are commonly used in food and beverage products. Non-limiting examples include pea protein, soy protein, alfalfa protein, golden flax protein, almond protein, cashew protein, canola (rapeseed) protein, chickpea protein, black bean protein, black eyed pea protein, northern bean protein, tobacco protein, mung bean protein, cowpea protein, pinto bean protein, fava bean protein, sunflower protein, quinoa protein, sesame protein, spelt protein, wheat protein, wheat berry protein, barley protein, wild rice protein, rice protein, oat protein, and potato protein. In some embodiments, the plant protein is a pea protein, such as pea protein extract or isolate. In some other embodiments, the plant protein is a soy protein, such as a soy protein extract or isolate.

It was discovered that the five of the taste receptors identified as SEQ ID NOS. 1-5 are each implicated in some way in connection with the human perception of the bitter taste of plant proteins. Therefore, in the introduction step (a) of either of the foregoing aspects, the taste receptor can comprise any combination of these polypeptide sequences. In some embodiments, the taste receptor comprises only one of the polypeptide sequences.

For example, in some embodiments, the taste receptor comprises a polypeptide sequence of SEQ ID NO: 1, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 1, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 2, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 2, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 3, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 3, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 4, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 4, or a polypeptide sequence whose sequence is at least 90% equivalent, or at least 95% equivalent, or at least 97% equivalent, thereto.

For example, in some embodiments, the taste receptor comprises a polypeptide sequence of SEQ ID NO: 5, a functional fragment thereof, or a polypeptide sequence whose sequence is at least 90% equivalent, or at least 95% equivalent, or at least 97% equivalent, to either of the foregoing. In some further embodiments, the taste receptor comprises: a polypeptide sequence of SEQ ID NO: 5, or a polypeptide sequence whose sequence is at least 90% equivalent, or at least 95% equivalent, or at least 97% equivalent, thereto.

In some embodiments of any of the foregoing embodiments, the introducing step (a) comprises introducing the test compound to any combination of two, three, four, or five of the polypeptide sequences identified above.

The initial introducing step (a) can be carried out in any suitable way. For example, in some non-limiting embodiments, when introducing a test compound to taste receptor, the introducing can be carried out in a single cell-based assay. Any suitable assay can be used, according to the knowledge of those skilled in the art.

In some embodiments, it can be desirable to measure the activity of the test compound in the presence of one or more additional compounds that are known to impart a bitter taste sensation. In this way, one can determine whether the test compound enhances or blocks (antagonizes) the binding of the one or more additional compounds. In some embodiments, the one or more additional compounds comprise a plant protein, such as any of the plant proteins set forth above. In some embodiments, the plant protein is a pea protein extract. Thus, in some embodiments, the introducing step (a) comprises introducing the test compound to the taste receptor in the presence of a plant protein.

The foregoing embodiments involve measuring a response of the taste receptor to the test compound. This measuring can be carried out by any suitable means. For example, in some embodiments, the taste receptor is expressed on the surface of cells, and compositions are screened against the cells expressing the taste receptor in a standard cellular assay. Measuring binding to the taste receptor can be carried out by any suitable means typically used in determining protein binding in cellular assays. Suitable methods include, but are not limited to, use of fluorescent dyes, a calcium indicator protein, a fluorescent calcium indicator, a fluorescent cAMP indicator, and the like. In some embodiments, activity of the test compound is determined by its binding to the taste receptor. In some embodiments, activity of the test compound is determined by its ability to block the binding of a bitter compound, such as a plant protein, to the taste receptor.

In some further embodiments of any of the foregoing embodiments, the methods further comprise identifying an active test compound that reduce or block bitter taste based on the measured response. According to the foregoing embodiments, the identified active test compound is one that modulates the taste receptor or antagonizes its modulation by a bitter compound, such as a plant protein.

In some embodiments of any of the foregoing embodiments, the test compound is determined to be an active compound because it modulates the taste receptor, for example, because it induces a response of the taste receptor having an EC50 of no more than 10 mM, or no more than 1 mM or no more than 500 μM, or no more than 250 μM, or no more than 100 μM, and because the test compound is not active on cells not expressing any of the polypeptide sequences of SEQ ID NOS. 1-5, or any functional fragments thereof.

In some embodiments of any of the foregoing embodiments, the test compound is determined to be an active compound because it modulates the activity of a bitter compound, such as a plant protein, to the taste receptor, for example, because it induces a response of the taste receptor having an EC50 of no more than 10 mM, or no more than, no more than 1 mM, no more than 500 μM, or no more than 250 μM, or no more than 100 μM in the presence of a bitter compound.

In some further embodiments, the methods further comprise selecting the active test compound as a compound that reduces a bitter taste, such as a bitter taste of a plant protein. The selecting step can be carried out by any suitable means once the active test compounds are identified.

The test compound can be any suitable compound that is amenable for use in cellular screening assays. For example, in some embodiments, the test compound is an organic compound. In some embodiments, the test compound is a naturally occurring compound. In some other embodiments, the test compound is a non-naturally occurring compound. In some embodiments, the test compound is a peptide, such as a polypeptide or an oligopeptide, an amino acid, an amino acid derivative, an amide, a nucleotide, an oligonucleotide. In some embodiments, the test compound is a plant or food extract, such as a plant or food extract obtained by bioassay-guided fractionation. In some embodiments, the test compound is a diterpene or a triterpene. In some embodiments, the test compound is a ribonucleotide or derivative thereof.

### Uses and Methods

In other aspects, the disclosure provides uses of any identified or selected active compounds of the foregoing aspects, including any embodiments or combination of embodiments thereof, as set forth above. In certain related aspects, the disclosure provides uses of any identified or selected active compounds of the foregoing aspects, including any embodiments or combination of embodiments thereof, as set forth above, to reduce a bitter taste of an ingestible composition, wherein the ingestible composition comprises one or more plant proteins.

The disclosure also provides methods that correspond to each of the foregoing uses. Thus, in certain related aspects, the disclosure provides methods of reducing a bitter taste of an ingestible composition, comprising introducing an amount (such as a bitter-reducing-effective amount) of any identified or selected active compounds of foregoing aspects, including any embodiments or combination of embodiments thereof, as set forth above, to the ingestible composition. In some embodiments, the ingestible composition comprises one or more plant proteins, such as pea protein.

The foregoing uses and methods generally involve the use of the identified or selected active compounds in a composition containing one or more additional ingredients. For example, in at least one aspect, the disclosure provides compositions comprising any identified or selected active compounds of the foregoing aspects, including any embodiments or combination of embodiments thereof, as set forth above, wherein the identified or selected active compounds make up at least 10% by weight, or at least 20% by weight, or at least 30% by weight, or at least 40% by weight, or at least 50% by weight, of the compositions on a dry weight basis (e.g., based on the total weight of the composition excluding the weight of any liquid carrier). In a related aspect, the disclosure provides solid-state compositions comprising any identified or selected active compounds of the foregoing aspects, including any embodiments or combination of embodiments thereof, as set forth above, wherein the identified or selected active compounds make up at least 10% by weight, or at least 20% by weight, or at least 30% by weight, or at least 40% by weight, or at least 50% by weight, of the solid-state compositions, based on the total weight of composition. In another related aspect, the disclosure provides ingestible compositions comprising identified or selected active compounds of the foregoing aspects, including any embodiments or combination of embodiments thereof, as set forth above, wherein the concentration of the identified or selected active compounds in the ingestible compositions is no more than 2500 ppm (such as from 1 ppm to 2500 ppm), or no more than 2000 ppm (such as from 1 ppm to 2000 ppm), or no more than 1500 ppm (such as from 1 ppm to 1500 ppm), or no more than 1000 ppm (such as from 1 ppm to 1000 ppm), or no more than 750 ppm (such as from 1 ppm to 750 ppm), or no more than 500 ppm (such as from 1 ppm to 500 ppm), or no more than 400 ppm (such as from 1 ppm to 400 ppm), or no more than 300 ppm (such as from 1 ppm to 300 ppm), or no more than 200 ppm (such as from 1 ppm to 200 ppm), or no more than 150 ppm (such as from 1 ppm to 150 ppm), or no more than 100 ppm (such as from 1 ppm to 100 ppm), or no more than 50 ppm (such as from 1 ppm to 50 ppm), or no more than 30 ppm (such as from 1 ppm to 30 ppm), or no more than 20 ppm (such as from 1 ppm to 20 ppm),

In certain embodiments of any aspects and embodiments set forth herein that refer to an ingestible composition, the ingestible composition is a non-naturally-occurring product or a naturally-occurring product, such as a composition specifically manufactured, extracted or purified for the production of a flavored product, such as food or beverage product.

In certain aspects, the disclosure provides ingestible compositions comprising one or more bitter compounds. In some embodiments, the bitter compound is a plant protein. Non-limiting examples include pea protein, soy protein, alfalfa protein, golden flax protein, almond protein, cashew protein, canola (rapeseed) protein, chickpea protein, black bean protein, black eyed pea protein, northern bean protein, tobacco protein, mung bean protein, cowpea protein, pinto bean protein, fava bean protein, sunflower protein, quinoa protein, sesame protein, spelt protein, wheat protein, wheat berry protein, barley protein, wild rice protein, rice protein, oat protein, and potato protein. In some embodiments, the plant protein is a pea protein, such as pea protein extract or isolate. In some other embodiments, the plant protein is a soy protein, such as a soy protein extract or isolate.

In general, identified or selected compounds, individually or in combination, can be provided in a composition, such as an ingestible composition. In one embodiment, compounds as disclosed and described herein, individually or in combination, can impart a more meat-like temporal profile or flavor profile to a composition by blocking the bitter taste indicative of plant proteins.

In some embodiments, such as when the ingestible composition is used in meat analogue products that use plant proteins, the ingestible composition includes one or more compounds that impart or enhance an umami or a kokumi taste. Such compounds include glutamates (such as monosodium glutamate), aspartates, inosine monophosphate, guanosine monophosphate, sodium salts (such as NaCl), potassium salts (such as KCl), yeast extracts, fermented food products, cheeses, garlic or extracts thereof, a polypeptide or an oligopeptide, an amino acid, an amino acid derivative, an amide, a nucleotide (such as, an oligonucleotide, or plant or food extract, such as a plant or food extract obtained by bioassay-guided fractionation. In some embodiments, the ingestible composition also includes one or more umami enhancers, such as naturally derived compounds, such as FEMA 4232, FEMA 4233, FEMA 4798, or FEMA 4877, or synthetic compounds, such as any compounds set forth in U.S. Pat. Nos. 8,735,081; 8,124,121; and 8,968,708. In some embodiments, the ingestible composition comprises a cinnamic acid amide, such as (E)-3-(3,4-dimethoxyphenyl)-N-(4-methoxyphenethyl) acrylamide, rubemamine, rubescenamine, or any combination thereof. In some embodiments, the ingestible composition comprises N-(heptan-4-yl)-benzo[d][1,3]dioxole-5-carboxamide, e.g., at a concentration ranging from 1 to 50 ppm.

In some embodiments, such as when the ingestible composition is used in dairy analogue products, the ingestible composition includes one or more compounds that impart or enhance sweet taste. Non-limiting examples of such compounds include caloric sweeteners, such as sucrose, fructose (for example, in the form of high-fructose corn syrup), glucose, allulose, galactose, maltose, lactose, sorbitol, xylitol, lactitol, mannitol, erythritol, maltitol, or any combinations thereof. Other non-limiting examples of such compounds include low-calorie sweeteners, such as aspartame, acesulfame potassium, saccharin, sucralose, neotame, advantame, steviol glycosides (such as rebaudiosode A, rebaudioside M, rebaudioside E, and the like), mogrosides (such as mogroside V, siamenoside I, isomogroside V, isomogroside V, mogroside IVe, the 1,6-α isomer of siamenoside I, and the like), and any combinations thereof. In some embodiments, the ingestible composition comprises sweetness enhancers. Such sweetness enhancing compounds include, but are not limited to, naturally derived compounds, such as hesperitin dihydrochalcone, hesperitin dihydrochalcone-4′-O′glucoside, neohesperitin dihydrochalcone, brazzein, hesperidin, phyllodulcin, naringenin, naringin, phloretin, glucosylated steviol glycosides, (2R,3R)-3-acetoxy-5,7,4′-trihydroxyflavanone, (2R,3R)-3-acetoxy-5,7,3′-trihydroxy-4′-methoxyflavanone, rubusosides, or synthetic compounds, such as any compounds set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1.6 bond, a β-1,2 bond, a β-1,4 bond, a β-1,6 bond, and so forth. In some embodiments of any of the preceding embodiments, the comestible composition comprises 3-((4-amino-2,2-dioxo-1H-benzo[c][1,2,6]thiadiazin-5-yl)oxy)-2,2-dimethyl-N-propyl-propanamide or N-(1-((4-amino-2,2-dioxo-1H-benzo[c][1,2,6]thiadiazin-5-yl)oxy)-2-methyl-propan-2-yl) isonicotinamide.

In some further embodiments, ingestible compositions disclosed herein comprise the one or more bitterness blocking compounds. Such bitterness blocking compounds include, but are not limited to, naturally derived compounds, or synthetic compounds, such as any compounds set forth in PCT Publication WO 2020/033669, and in U.S. Pat. Nos. 8,076,491; 8,445,692; and 9,247,759.

In some embodiments, the ingestible composition comprises one or more fats. The fat can be any suitable fat, such as a fat derived from an animal or vegetable fat, including, but not limited to, milk fat (including fat in various cheeses), beef fat, pork fat, poultry fat, lamb fat, goat fat, fish oil, olive oil, canola oil, corn oil, safflower oil, nut oil, peanut oil, cashew oil, soybean oil, palm oil, palm kernel oil, coconut oil, butter, and nut butters (such as peanut butter, cashew butter, almond butter, hazelnut butter, and the like).

In some embodiments, the ingestible compositions disclosed herein comprise one or more mouthfeel modifying compounds. Such mouthfeel modifying compounds include, but are not limited to, tannins, cellulosic materials, bamboo powder, and the like.

In some embodiments, ingestible compositions disclosed herein comprise one or more flavor masking compounds. Such flavor masking compounds include, but are not limited to, cellulosic materials, materials extracted from fungus, materials extracted from plants, citric acid, carbonic acid (or carbonates), and the like.

The ingestible compositions can, in certain embodiments, comprise any additional ingredients or combination of ingredients as are commonly used in food and beverage products, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, Panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions can have any suitable pH. In some embodiments, the flavor-modifying compounds enhance the sweetness of a sweetener under a broad range of pH, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7. In certain embodiments, compounds as disclosed and described herein, individually or in combination, can enhance the perceived sweetness of a fixed concentration of a sweetener in taste tests at a compound concentration of 50 μM, 40 μM, 30 μM, 20 μM, or 10 UM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

### Flavored Products

In certain aspects, the disclosure provides flavored products comprising any compositions of the preceding aspects. In some embodiments, the flavored products are beverage products, such as a protein drink, a meal replacement beverage, and the like. In some other embodiments, the flavored products are food products, such as a dairy analogue or a meat analogue.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

In some embodiments, the flavored product includes one or more adjuvants. The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

### Non-Animal Protein Materials and Products Made Therefrom

Products intended to replace or substitute meat or dairy products often rely on various non-animal-based materials, such as starches and proteins derived from plants, algae, and fungi, to simulate the texture and flavor of meat or dairy. Non-limiting examples of such plant proteins include soy proteins, pea proteins, bean proteins, grain proteins, and the like. Due to compositional differences between such plant-based materials and animal-derived materials, such as a lack of glutamate-containing proteins and glutathione, these products can lack the umami and/or kokumi taste that consumers traditionally associate with meat or dairy products.

Thus, in certain aspects, the disclosure provides a flavored product comprising a plant-based material (such as a plant-based starch, a plant-based protein, or a combination thereof) and the active compound, or a comestibly acceptable salt thereof. In some further embodiments, the flavored product can include any features of combination of features set forth above for ingestible compositions that contain the active compound, or a comestibly acceptable salt thereof. In some embodiments, the flavored product is a beverage, such as soy milk, almond milk, rice milk, oat milk, a protein drink, a meal-replacement drink, or other like product. In some other embodiments, the flavored product is a meat-replacement product, such as a plant-based chicken product (such as a plant-based chicken nugget), a plant-based beef product (such as a plant-based burger), and the like. In some other embodiments, the flavored product is a protein powder, a meal-replacement powder, a plant-based creamer for coffee or tea, and the like. In certain further embodiments, any such products contain additional ingredients, and have additional features, as are typically used in the preparation and/or manufacture of such products. For example, such the active compound, or comestibly acceptable salts thereof, may be combined with other flavors and taste modifiers, and may even be encapsulated in certain materials, according to known technologies in the relevant art. Suitable concentrations of the active compound, or comestibly acceptable salts thereof, are set forth above.

In some further embodiments analogous to the above embodiments, proteins or starches from algal or fungal sources can be used instead of or in combination with plant starches or proteins.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1—Assays

Cells expressing a promiscuous G protein (G16g44) were transfected using TransITwith expression vectors encoding 22 functional biter receptor cDNAs, with one receptor per transfection. The cells were transfected directly in 384-well format high-density plates and incubated for 28 hours. Later, cells were loaded with the Ca-specific dye Fluo4-AM. After an incubation of 60 minutes, excess dye is washed off, and cells are stimulated with respective bitter agonists while simultaneously recording changes in intracellular fluorescence using a FLIPR instrument. Bitter agonist and responding receptors were identified.

The above protocol was carried out using fractions from pea and soy protein extract as a non-limiting example of a bitter agonist. Data shows that Pea and Soy fractions activate hT2R10, hT2R14 or hT2R61.

### Example 2—Bitterness Antagonism

Cpd A (50 μM) inhibits hT2R10, hT2R14 and hT2R61 in the assay. Cpd B (50 μM) inhibits hT2R10 and hT2R61 in the assay.

### Example 3—Taste Tests

Using business standard taste it was determined that Cpd A and Cpd B attenuate bitterness of Pea protein in human panelists.

1. A method of identifying compounds that reduce a bitter taste of a plant protein, the method comprising:
(a) introducing a test compound to a taste receptor, which comprises a polypeptide sequence of SEQ ID NO: 1, SEQ ID NO: 2, SEQ ID NO: 3, SEQ ID NO: 4, SEQ ID NO: 5, a functional fragment of any of the foregoing, or a polypeptide sequence whose sequence is at least 90% equivalent to any of the foregoing; and
(b) measuring a response of the taste receptor to the test compound by comparing an activity of the taste receptor in the presence and the absence of the test compound.

2. A method of identifying compounds that reduce bitter taste sensation, the method comprising:
(a) introducing a test compound to a taste receptor in the presence of a plant protein, wherein the taste receptor comprises a polypeptide sequence of SEQ ID NO: 1, SEQ ID NO: 2, SEQ ID NO: 3, SEQ ID NO: 4, SEQ ID NO: 5, a functional fragment of any of the foregoing, or a polypeptide sequence whose sequence is at least 90% equivalent to any of the foregoing; and
(b) measuring a response of the taste receptor to the test compound by comparing an activity of the taste receptor in the presence and the absence of the test compound.

3. The method of claim 1 or 2, further comprising: (c) identifying an active test compound that reduces the bitter taste of the plant protein based on the measured response.

4. The method of claim 3, further comprising (d) selecting the active test compound as a compound that reduces the bitter taste of the plant protein.

5. The method of claim any one of claims 2 to 4, wherein the plant protein is a pea protein, a soy protein, or a combination thereof.

6. The method of claim 5, wherein the plant protein is a pea protein.

7. The method of any one of claims 1 to 6, wherein the test compound is a naturally occurring compound.

8. The method of any one of claims 1 to 6, wherein the test compound is a non-naturally occurring compound.

9. Use of a taste-modifying compound to reduce a bitter taste of an ingestible composition, wherein the taste-modifying compound is the identified or selected compound of any one of claims 3 to 8.

10. The use of claim 9, wherein the taste-modifying compound is a naturally occurring compound,

11. The use of claim 9, wherein the taste-modifying compound is a non-naturally occurring compound.

12. The use of any one of claims 9 to 11, wherein the ingestible composition comprises a plant protein.

13. The use of any one of claims 9 to 12, wherein the ingestible composition further comprises a sweetener, an umami compound, a mouthfeel enhancer, a sweetness enhancer, an umami enhancer, or any combination thereof.

14. An ingestible composition comprising a plant protein and a taste-modifying compound, wherein the taste-modifying compound is the identified or selected compound of any one of claims 3 to 8.

15. The ingestible composition of claim 14, wherein the plant protein is a pea protein, a soy protein, or any combination thereof.

16. The ingestible composition of claim 14 or 15, further comprising a sweetener, an umami compound, a mouthfeel enhancer, a sweetness enhancer, an umami enhancer, or any combination thereof.

17. A flavored product, which comprises an ingestible composition of any one of claims 14 to 16.

18. The flavored product of claim 17, which is a dairy analogue or a meat analogue.
